(19) United States
(12) Patent Application Publication
(10) Pub. No.: US 2025/0025363 A1
(43) Pub. Date: Jan. 23, 2025

(54) INTELLIGENT REHABILITATION DEVICE

(71) Applicant: XI'AN LIBANG CONTMEDU MEDICAL TECHNOLOGY CO., LTD., Xi'an (CN)

(72) Inventor: Changping Qu, Xi'an (CN)

(21) Appl. No.: 18/776,370

(22) Filed: Jul. 18, 2024

Related U.S. Application Data
(63) Continuation-in-part of application No. PCT/CN2024/088234, filed on Apr. 17, 2024.

(30) Foreign Application Priority Data
Apr. 17, 2023 (CN) 202310411659.5
Apr. 17, 2023 (CN) 202320857876.2
Mar. 27, 2024 (CN) 202410354962.0
Apr. 8, 2024 (CN) 202410411544.0

(51) Int. Cl. A61H 1/02; G16H 20/30; G16H 40/67; G16H 50/20

(52) U.S. Cl. CPC A61H 1/0237; G16H 20/30; G16H 40/67; G16H 50/20; A61H 2201/165

(57) ABSTRACT

Embodiments of the present disclosure provide an intelligent rehabilitation device, which can solve problems of low intelligence in use of a rehabilitation device and low efficiency in rehabilitation management. The intelligent rehabilitation device includes a mobile cart, and an embedded upper computer, a mobile unit, and a wearable unit that are installed on the mobile cart. The embedded upper computer is equipped with a rehabilitation training module, a rehabilitation assessment module, and a mobile control module. The mobile unit includes a roller, an electric push rod, and a driver that are disposed on the mobile cart. The wearable unit includes a rehabilitation training glove and a somatosensory sensor, and both the rehabilitation training glove and the somatosensory sensor are configured to perform rehabilitation training through the rehabilitation training module.

### CROSS REFERENCE TO RELATED APPLICATIONS

The present application is a Continuation-In-Part application of PCT Application No. PCT/CN2024/088234 filed on Apr. 17, 2024, which claims the benefit of Chinese Patent Application Nos. 202310411659.5 filed on Apr. 17, 2023, 202320857876.2 filed on Apr. 17, 2023, 202410354962.0 filed on Mar. 27, 2024 and 202410411544.0 filed on Apr. 8, 2024. All the above are hereby incorporated by reference in their entirety.

### TECHNICAL FIELD

Embodiments of the present disclosure relate to the technical field of medical treatment, and specifically, to an intelligent rehabilitation device.

### BACKGROUND

With the improvement of people's living standards and the intensification of population aging, cardiovascular and cerebrovascular diseases have become a health killer, and stroke is a leading factor causing death and lasting disability. Clinical research and practice have verified that through timely and effective rehabilitation training, about 90% of patients can recover from a certain daily exercise and self-care capability. In clinical practice, a period from 0 to 6 months is known as a golden rehabilitation period, but rehabilitation is a long-term process, and mostly should last for more than one year. In addition, there are a large number of stroke patients, and rehabilitation medical resources are very scarce, resulting in a great demand for an intelligent management device.

At present, rehabilitation therapy is usually adjuvant therapy manually conducted by rehabilitation therapists or one-on-one treatment with mechanical assistive devices for patients. This method has many shortcomings: (1) Treatment efficiency is low, and a labor cost is high. (2) Rehabilitation assessment has strong subjectivity, which makes it impossible to guarantee accuracy of a rehabilitation training assessment result. (3) Selected training actions are not closely related to daily life, making it difficult for patients to return to normal social life. (4) A fixed position and height of the device make it inconvenient for patients who cannot move around.

### SUMMARY

Embodiments of the present disclosure provide an intelligent rehabilitation device, which can improve rehabilitation treatment efficiency of patients and make rehabilitation management devices more intelligent.

Technical solutions provided in the embodiments of the present disclosure are as follows:

An intelligent rehabilitation device includes a mobile cart, where
the mobile cart includes an embedded upper computer, a mobile unit, and a wearable unit, and both the mobile unit and the wearable unit are communicatively connected to the embedded upper computer;
the embedded upper computer includes a rehabilitation training module, a rehabilitation assessment module, a data collection module, a data processing module, a mobile control module, and a storage module;
the rehabilitation training module is configured to provide a scene training game designed based on a rehabilitation action, and the scene training game is used to train an upper limb, a lower limb, and a hand of a rehabilitation training patient;
the rehabilitation assessment module is configured to assess movement functions of the upper limb, the lower limb, and the hand of the rehabilitation training patient;
the data collection module is configured to collect information of the rehabilitation training patient, including feature vectors corresponding to a plurality of dimensions of the patient, where each feature vector is used to indicate patient information of each dimension, and the dimensions include a medical record, a lifestyle habit, an environmental factor, and a rehabilitation exercise of the patient;
the data processing module is configured to: predict an impact of the patient information of each dimension on rehabilitation of the patient based on the feature vector of each dimension, obtain a first prediction vector corresponding to the feature vector of each dimension, and perform feature concatenation on each first prediction vector to obtain a target vector, where the first prediction vector is used to indicate the impact of the patient information of each dimension on the rehabilitation of the patient; predict, based on the target vector, a rehabilitation effect corresponding to the patient information of each dimension, and obtain a second prediction vector, where the second prediction vector is used to indicate the rehabilitation effect corresponding to the patient information of each dimension; and generate prescription information for the rehabilitation of the patient based on the second prediction vector, where the prescription information includes medication information and a medication dosage;
the mobile control module is configured to control, by obtaining a position of the rehabilitation training patient and measuring a distance from the rehabilitation training patient, the mobile cart to move and adjust a height;
the mobile unit includes a roller, an electric push rod, and a driver that are disposed on the mobile cart, where the driver is connected to the mobile control module, and through the driver, the mobile control module drives the roller to move and adjust a position, and controls the electric push rod to adjust a height; and
the wearable unit includes a rehabilitation training glove and a somatosensory sensor, where both the rehabilitation training glove and the somatosensory sensor are configured to perform rehabilitation training through the rehabilitation training module and send rehabilitation training data to the data collection module.

Further, the data collection module includes a receiver and a camera, where the receiver is wirelessly connected to both the rehabilitation training glove and the somatosensory sensor;
the receiver is configured to receive movement data from the rehabilitation training glove and the somatosensory sensor, and the camera collects the movement data by capturing an action of the rehabilitation training patient; and
the data processing module is further configured to process the movement data based on multi-source information fusion.

Further, the intelligent rehabilitation device includes: a human-machine interaction column equipped with a card reading area and a button area, where the card reading area is configured to recognize an identity of the rehabilitation training patient, and the button area is equipped with a plurality of buttons to assist in the scene training game;
a cloud server, where the cloud server stores an exercise prescription; and
the embedded upper computer is configured to download the exercise prescription or upload the rehabilitation training data through the cloud server; and
a power module, where the power module includes a magnetic ring, a transformer, a filter, and a data line, and is configured to provide a power supply and process a power signal.

Further, the rehabilitation training glove includes a glove body and a first housing installed on the glove body; and the first housing is equipped therein with a first battery, a first circuit board, and a bending sensor, and a first indicator light is installed on the first housing.

Further, the somatosensory sensor includes a bandage and a second housing installed on the bandage; and the second housing is equipped therein with a second circuit board and a second battery, and a second indicator light is installed on the second housing.

Further, the rehabilitation assessment module includes an assessment assistance unit and an assessment unit, where the assessment unit includes an intelligent scale assessment subunit and a compensatory-movement quantitative assessment subunit;
the assessment assistance unit is configured to complete a task of recognizing a rehabilitation assessment action and obtain a recognition result; and
the assessment unit is configured to obtain a corresponding assessment result based on the recognition result.

Further, the embedded upper computer is interconnected with a terminal through a network, and configured for remote medical diagnosis or online rehabilitation training guidance.

Further, the mobile control module is connected to a radar detector and a positioning module; the radar detector is configured to detect the position of the rehabilitation training patient; and the positioning module is configured to determine a current position of the mobile cart.

Further, the mobile cart includes a base and a connecting piece, where the roller is disposed at a bottom of the base, the electric push rod is disposed inside the connecting piece, and the embedded upper computer is connected to a top of the electric push rod.

Further, the mobile control module is connected to an infrared sensor and an infrared signal transmitter, and when the intelligent rehabilitation device is in use, position and height adjustment is achieved through the infrared sensor, the infrared signal transmitter, and the mobile control module.

Compared with the prior art, the embodiments of the present disclosure have following beneficial effects:
1. The embodiments of the present disclosure provide an intelligent rehabilitation device, including a mobile cart, and an embedded upper computer, a mobile unit, and a wearable unit that are installed on the mobile cart. The mobile unit is controlled by the embedded upper computer to achieve intelligent position and height recognition and height adjustment.
2. The intelligent rehabilitation device provided in the embodiments of the present disclosure executes a rehabilitation training task in a form of a virtual game through a rehabilitation training module in the embedded upper computer, and obtains a visualized quantitative rehabilitation assessment result through a rehabilitation assessment module. This achieves a simple operation, a high work efficiency, and high entertainment value.
3. The intelligent rehabilitation device provided in the embodiments of the present disclosure can perform multidirectional rehabilitation training on an upper limb, a lower limb, and a finger through a rehabilitation training glove and a somatosensory sensor, and accurately assess a rehabilitation training action by recognizing the rehabilitation training action and establishing scale assessment and compensation assessment. Compared with the existing assessment system and device, the intelligent rehabilitation device achieves a more accurate assessment result. In addition, the assessment model can also perform learning and has a broad application market.
4. The intelligent rehabilitation device provided in the embodiments of the present disclosure establishes a rehabilitation record through a human-machine interaction column, keeping track of rehabilitation progress of each patient at any time. This not only improves user experience, but also reduces a burden on staff through detailed record management, making an entire rehabilitation training process more standardized.
5. The intelligent rehabilitation device provided in the embodiments of the present disclosure loads different types of rehabilitation training games through the rehabilitation training module, and selects an interesting small game that is closely related to daily life to assist the patient in rehabilitation training. Through the rehabilitation training module, the upper limb, the lower limb, and the finger of the patient can be fully trained, accelerating rehabilitation progress of the patient.
6. The intelligent rehabilitation device provided in the embodiments of the present disclosure can predict a rehabilitation effect of the patient by obtaining patient information of each dimension, and generate prescription information for rehabilitation of the patient based on the patient information of each dimension, thereby improving rehabilitation efficiency of the patient.

### BRIEF DESCRIPTION OF THE DRAWINGS

FIG. 1 is a first structural diagram of an intelligent rehabilitation device according to an embodiment of the present disclosure;

FIG. 2 is a second structural diagram of an intelligent rehabilitation device according to an embodiment of the present disclosure;

FIG. 3 is a third structural diagram of an intelligent rehabilitation device according to an embodiment of the present disclosure;

FIG. 4 is a schematic diagram of an intelligent rehabilitation device according to an embodiment of the present disclosure;

FIG. 5 is a structural diagram of a rehabilitation training glove of an intelligent rehabilitation device according to an embodiment of the present disclosure;

FIG. 6 is a structural diagram of a somatosensory sensor of an intelligent rehabilitation device according to an embodiment of the present disclosure; and

FIG. 7 is a fourth structural diagram of an intelligent rehabilitation device according to an embodiment of the present disclosure.

### REFERENCE NUMERALS IN THE ACCOMPANYING DRAWINGS

1: mobile cart; 101: base; 102: connecting piece; 11: embedded upper computer; 111: data collection module; 1111: receiver; 1112: camera; 112: data processing module; 113: mobile control module; 114: rehabilitation training module; 115: rehabilitation assessment module; 116: storage module; 12: mobile unit; 121: driver; 122: electric push rod; 123: roller; 2: wearable unit; 201: rehabilitation training glove; 202: somatosensory sensor; 13: human-machine interaction column; 14: power module.

### DETAILED DESCRIPTION OF THE EMBODIMENTS

To make the objective, technical solutions, and advantages of the present disclosure clearer, the technical solutions in the embodiments of the present disclosure are clearly and completely described below with reference to the accompanying drawings in the embodiments of the present disclosure. Apparently, the described embodiments are some rather than all of the embodiments of the present disclosure. Generally, the components shown in the accompanying drawings of the embodiments of the present disclosure may be provided and designed in various manners.

Therefore, the detailed description of the embodiments of the present disclosure provided in conjunction with the accompanying drawings is intended to represent only the selected embodiments of the present disclosure and does not limit the protection scope claimed by the present disclosure. All other embodiments obtained by those of ordinary skill in the art based on the embodiments of the present disclosure without creative efforts should fall within the protection scope of the present disclosure.

It should be understood that, in the description of the implementations in the embodiments of the present disclosure, the terms such as “first” and “second” are only for the purpose of description and should not be construed as indicating or implying relative importance, or implicitly indicating a quantity of indicated technical features. Therefore, features defined by “first” and “second” can explicitly or implicitly include one or more of the features.

In the description of the implementations in the embodiments of the present disclosure, it should be noted that unless otherwise expressly specified, terms such as “disposed”, “installed”, “connected to”, and “connected with” should be comprehended in a broad sense. For example, the “connection” may be a fixed connection, a removable connection, or an integral connection; may be a mechanical connection, an electrical connection, or mutual communication; may be a direct connection or an indirect connection via an intermediate medium; or may be an interconnection or an interaction relationship between two elements. Those of ordinary skill in the art may understand specific meanings of the foregoing terms in the implementations in the embodiments of the present disclosure based on a specific situation.

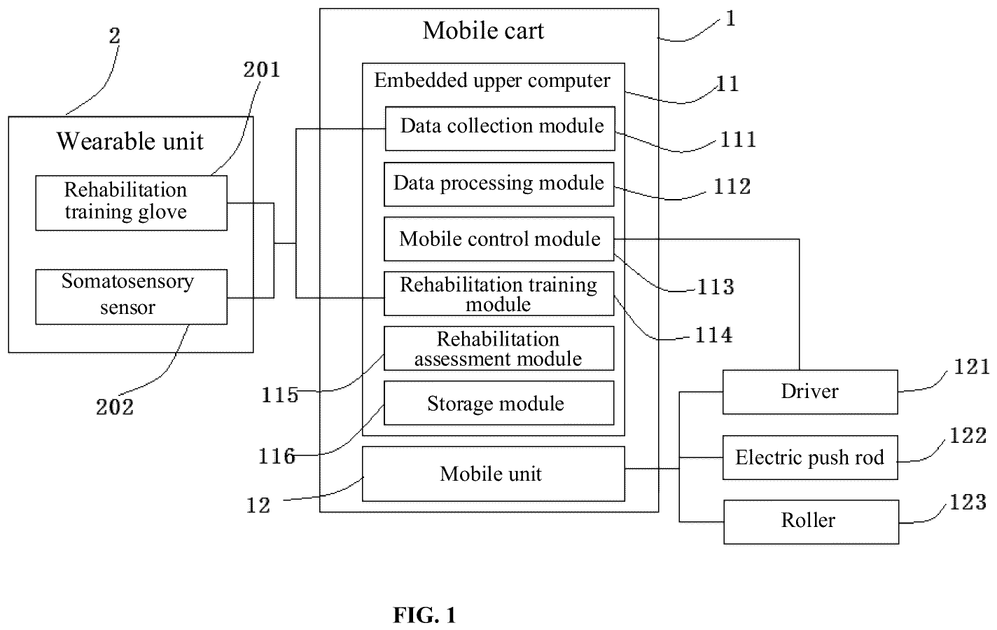

FIG. 1 is an overall structural diagram of an intelligent rehabilitation device according to an embodiment of the present disclosure. Referring to FIG. 1, the embodiments of the present disclosure provide an intelligent rehabilitation device, including:
a mobile cart 1, and an embedded upper computer 11, a mobile unit 12, and a wearable unit 2 that are installed on the mobile cart 1, where both the mobile unit 12 and the wearable unit 2 are communicatively connected to the embedded upper computer 11.

The embedded upper computer 11 includes a processor and a memory. The processor is configured to execute programs of the following program modules stored in the memory: a data collection module 111, a data processing module 112, a mobile control module 113, a rehabilitation training module 114, and a rehabilitation assessment module 115.

The processor may be a central processing unit (CPU), a microprocessor, an application-specific integrated circuit (ASIC), or one or more integrated circuits configured to execute a program of the solutions in the present disclosure.

The memory may be a storage module 116 in the present disclosure. The memory may be a read-only memory (ROM), another type of static storage device that can store static information and instructions, a random access memory (RAM), another type of dynamic storage device that can store information and instructions, an electrically erasable programmable read-only memory (EEPROM), a disk storage medium, another magnetic storage device, or any other medium that can be used to carry or store desired program code in a form of an instruction or a data structure and can be accessed by a computer, but is not limited thereto.

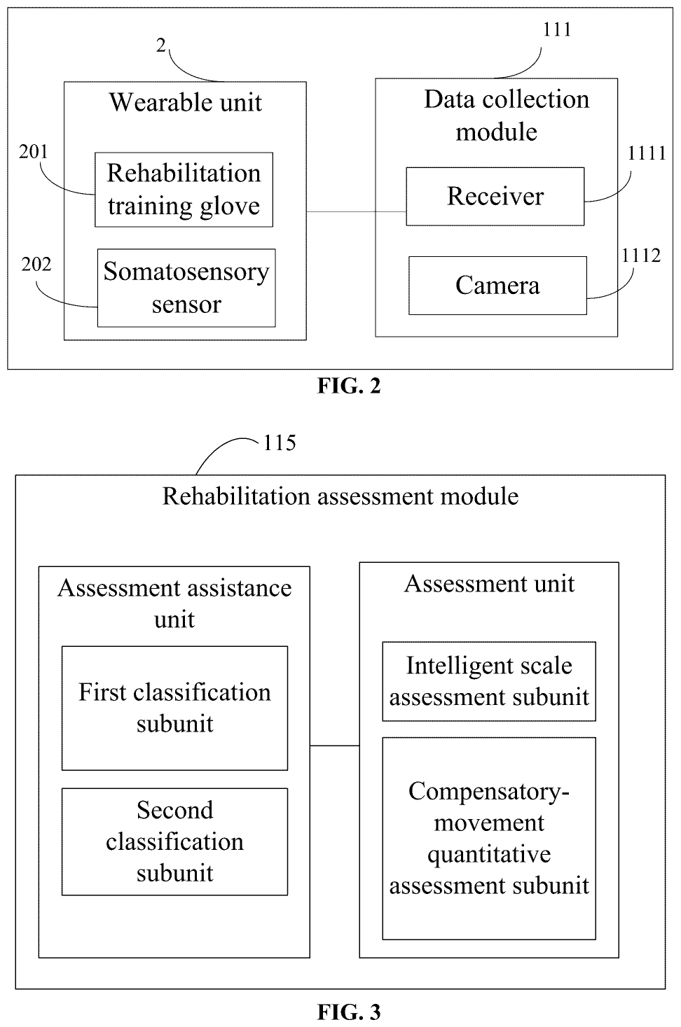

As shown in FIG. 2, the wearable unit 2 includes a rehabilitation training glove 201 and a somatosensory sensor 202. Both the rehabilitation training glove 201 and the somatosensory sensor 202 are configured to perform rehabilitation training through the rehabilitation training module 114 and send rehabilitation training data to the data collection module 111. The wearable unit 2 communicates wirelessly with the embedded upper computer 11 through a ZigBee, Bluetooth, or wireless fidelity (Wi-Fi) wireless communication protocol, such that the wearable unit 2 can perform the rehabilitation training based on training content of the rehabilitation training module 114 and send the rehabilitation training data to the data collection module 111.

The data collection module 111 is configured to collect information of a rehabilitation training patient, including feature vectors corresponding to a plurality of dimensions of the patient. Each feature vector is used to indicate patient information of each dimension, and the dimensions include a medical record, a lifestyle habit, an environmental factor, and a rehabilitation exercise of the patient.

The medical record of the patient includes: a patient complaint, a current medical history, a past medical history, a personal life history, a marital and childbearing history, a family history, a diagnosis record, a treatment recommendation, a surgical record, a pathological examination report, and the like. The lifestyle habit includes a diet, an exercise status, a smoking history, an alcohol consumption, a daily routine, and the like. The environmental factor includes temperature, humidity, air dust, wind power, a decibel of environmental noise, air quality, and the like. The rehabilitation exercise includes a movement function, a balance function, and a cognitive function. Certainly, in addition to factors of these dimensions, there are other factors that can affect rehabilitation of the patient, such as a gender, a place of residence, an age, an education level, a family condition, the marital and childbearing history, an economic status, a career, a job type, and a work environment. Taking the lifestyle habit as an example, including the diet (normal: 0; salty: 1; light: 2; heavy oil: 3, . . . , and so on), whether to make an exercise (moderate exercise: 0; never exercise: 1; gentle exercise: 2; plenty of exercise: 3; excessive exercise: 4, . . . , and so on), whether to smoke, whether to drink alcohol, and whether to have a regular daily routine, a feature vector of the lifestyle habit is established, namely (0, 0, 1, 1, 0, 0 . . . ).

The data processing module 112 includes a first prediction unit, a processing unit, a second prediction unit, and a generation unit. The first prediction unit is configured to: predict an impact of the patient information of each dimension on the rehabilitation of the patient based on the feature vector of each dimension, obtain a first prediction vector corresponding to the feature vector of each dimension, where the first prediction vector is used to indicate the impact of the patient information of each dimension on the rehabilitation of the patient. The processing unit is configured to perform feature concatenation on each first prediction vector to obtain a target vector. The second prediction unit is configured to predict, based on the target vector, a rehabilitation effect corresponding to the patient information of each dimension, and obtain a second prediction vector, where the second prediction vector is used to indicate the rehabilitation effect corresponding to the patient information of each dimension. The generation unit is configured to generate prescription information for the rehabilitation of the patient based on the second prediction vector, where the prescription information includes medication information and a medication dosage.

In the embodiments of the present disclosure, the processor may be configured to execute programs of the following program units stored in the memory: the first prediction unit, the processing unit, the second prediction unit, and the generation unit.

The prescription information also includes a medical institution name, a patient name, a gender, and an age, an outpatient or inpatient medical record number, a department or a ward, a bed number, a clinical diagnosis, an issuance date, a name, a dosage form, a specification, a quantity, usage and dosage of a drug, and the like.

Optionally, the first prediction unit is specifically configured to input the feature vector corresponding to each dimension into a first prediction model corresponding to each dimension to obtain the first prediction vector corresponding to each feature vector. The first prediction vector is used to indicate the impact of the patient information of each dimension on the rehabilitation of the patient. Each first prediction model is a neural network model based on a long short-term memory (LSTM), and the neural network model based on the LSTM is a model trained using a plurality of patient information samples and corresponding label information.

The second prediction unit is specifically configured to input the target vector into a preset second prediction model to obtain the second prediction vector corresponding to each first prediction vector. The second prediction vector is used to indicate a prediction result of the rehabilitation effect corresponding to the patient information of each dimension. The second prediction model is a neural network model based on an attention mechanism. The neural network model based on the attention mechanism is a model trained using a plurality of information samples of the impact of the patient information of each dimension on the rehabilitation of the patient and corresponding label information.

The processing unit is further configured to: obtain an activation function including a weight coefficient of each dimension and a preset bias constant, input the second prediction vector into the activation function, and use the activation function to determine at least one target sub-vector from the second prediction vector.

The generation unit is further configured to generate the prescription information for the rehabilitation of the patient based on the at least one target sub-vector.

The processing unit is further configured to: obtain a rehabilitation prescription atlas database including a plurality of pieces of prescription information; generate a corresponding prescription vector based on each piece of prescription information in the rehabilitation prescription atlas database; obtain a matching function and a weight vector, where the matching function is used to match a corresponding prescription vector based on the target sub-vector and the weight vector; and construct a prescription generation model based on the prescription vector, the matching function, and the weight vector, where the weight vector is a preset parameter of the model.

The processing unit is further configured to: obtain predicted prescription information generated by the prescription generation model for a plurality of patients, and obtain actual prescription information corresponding to each piece of predicted prescription information, where the actual prescription information includes actual medication information of the patient; obtain a loss function of the prescription generation model; and train the prescription generation model using each piece of predicted prescription information and the corresponding actual prescription information until a threshold of the loss function of the prescription generation model meets a preset condition, to obtain a target prescription generation model.

The generation unit is specifically configured to input the at least one target sub-vector into the target prescription generation model to obtain the prescription information for the rehabilitation of the patient.

Specifically, the second prediction unit can arbitrarily choose to output predicted impacts of the lifestyle habit, the environmental factor, rehabilitation training, and other factors on the rehabilitation effect through a multi-output control strategy. The multi-output control strategy is as follows:

$$H ( x , W , V , b , c ) = σ ( xW + b ) ( xV + c )$$

As described above, σ represents the activation function, which is a sigmoid function or another function by default and selected based on actual needs, x represents the second prediction vector, W and V represent weight coefficients, and b and c represent bias constants.

The loss function of the prescription generation model is as follows:

$${min}_{{x}_{*} , {y}_{*}} L ( X , Y ) = {min}_{{x}_{*} , {y}_{*}} {\sum }_{u , i} {( {r}_{ui} - {x}_{u}^{T} {y}_{i} )}^{2} + γ ( {\vert {x}_{u} \vert}^{2} + {\vert {y}_{i} \vert}^{2} )$$

As described above, x_{*} represents an optimal parameter of a predicted prescription, y_{*} represents an optimal parameter of an actual prescription, X, Y in L (X, Y) respectively represent a predicted prescription vector and an actual prescription vector, u represents a serial number of the predicted prescription vector, i represents a serial number of the actual prescription vector, xᵤ^{T} represents a transpose vector of a dense vector of a u^{th} predicted prescription vector, yᵢ represents a dense vector of an i^{th} actual prescription vector, rᵤᵢ represents an element in an interaction matrix of the predicted prescription vector and the actual prescription vector, where the element includes a correlation or a distance, xᵤ represents the dense vector of the u^{th} predicted prescription vector, and y represents a bias coefficient.

Further, in a model training process, the second prediction unit can also improve training efficiency of the model by a reward and punishment system and an error feedback method. The reward and punishment system includes:

$${w}_{ij} = \frac{{\sum }_{m ∈ N ( i ) ⋂ N ( j )} \frac{l}{log ( l + \vert N ( i ) \vert )}}{\sqrt{N ( i ) N ( j )}}$$

As described above, i and j represent dimension serial numbers, m represents an intersection between a predicted prescription vector and a corresponding actual prescription vector, N(i) represents an i^{th} predicted prescription information vector, N(j) represents a j^{th} actual prescription information vector, and l represents a reward and punishment constant.

The error feedback method includes:

$$RMSE = \sqrt{\frac{1}{N} {\sum }_{t}^{N} {( {observed}_{t} - {predicted}_{t} )}^{2}}$$

As described above, t represents a dimension serial number, observed, represents a t^{th} piece of actual prescription information, predicted represents a t^{th} piece of predicted prescription information, and N represents a dimension of the feature vector.

In addition, in order to improve visualization of rehabilitation execution management of the patient, the processing unit can also observe an execution status of the patient through compliance calculation.

$${Compliance}_{i} = {\sum }_{n = 0}^{N} {Task}_{n} * {Excute}_{n}$$

As described above, Task represents a predicted prescription information vector, Excute represents an actual prescription information vector, Complianceᵢ represents expected execution force of an i^{th} piece of predicted prescription information, and n represents a serial number of an information vector.

$$Compliance = {\sum }_{i} {Compliance}_{i} * {w}_{i}$$

As described above, wᵢ represents a weight vector of the i^{th} piece of predicted prescription information. Finally, an output is provided in a form of an executive force atlas of the patient and a comprehensive calculation value of a complex factor.

The mobile control module 113 is configured to control, by obtaining a position of the rehabilitation training patient and measuring a distance from the rehabilitation training patient, the mobile cart 1 to move towards the rehabilitation training patient and adjust a height to adapt to rehabilitation training patients of different heights. This achieves better user experience, and provides great convenience for patients with mobility difficulties.

The rehabilitation training module 114 is configured to provide a scene training game designed based on a rehabilitation action, and the scene training game is used to train an upper limb, a lower limb, and a hand of the rehabilitation training patient.

The rehabilitation assessment module 115 is configured to assess movement functions of the upper limb, the lower limb, and the hand of the rehabilitation training patient. After the rehabilitation training module 114 completes the rehabilitation training, the rehabilitation assessment module 115 assesses a training result by means of scoring.

Optionally, as shown in FIG. 3, the rehabilitation assessment module 115 includes an assessment assistance unit and an assessment unit. The assessment assistance unit is configured to complete a task of recognizing a rehabilitation assessment action. The assessment assistance unit is a cascaded model consisting of two parts, in other words, includes a first classification subunit and a second classification subunit. The assessment unit includes an intelligent scale assessment subunit and a compensatory-movement quantitative assessment subunit.

In the embodiments of the present disclosure, the processor may be configured to execute programs of the following program units stored in the memory: the assessment assistance unit, the assessment unit, the first classification subunit, the second classification subunit, the intelligent scale assessment subunit, and the compensatory-movement quantitative assessment subunit.

The first classification subunit adopts a neural network structure to recognize whether a rehabilitation assessment action that the patient is completing is an upper arm action, a palm action, a lower limb action, or an upper limb action. An input of a neural network is a movement feature vector (a vector obtained by determining a threshold of an original signal of the wearable unit), and an output is one of the four action categories mentioned above. The neural network includes an input layer with a quantity of neurons equal to a length of the movement feature vector, which is preset to N. The network includes two hidden layers, with 2N neurons in a first hidden layer and N neurons in a second hidden layer. The network includes an output layer with four neurons. Finally, the network completes a final output through a softmax layer and one-hot encoding. A calculation method of the network is as follows:

$${a}_{j} = σ ( {\sum }_{i = 1}^{N} {w}_{ij} {x}_{i} + {b}_{j} )$$

$${Z}_{k} = σ ( {\sum }_{i = 1}^{N} {w}_{jk} {a}_{j} + {b}_{k} )$$

$$output = ont - hot ( soft max ( z ) )$$

As described above, aⱼ represents an output of the hidden layer, σ represents the activation function, N represents the quantity of neurons, i represents a loop parameter, wᵢⱼ represents a weight, xᵢ represents an input, bⱼ represents a bias parameter, Zₖ represents a kth output of the output layer, wⱼₖ represents a weight, bₖ represents a bias parameter, output represents the final output, ont-hot represents an encoding function, and soft max (z) represents the activation function.

The second classification subunit adopts a gated recurrent network to recognize a specific rehabilitation assessment action. An input is an original signal of a wearable device of the intelligent rehabilitation device, and an output is a specific recognition result. A calculation method is as follows:

$${g}_{r} = σ ( {W}_{r} [ {h}_{t - 1} , {x}_{t} ] + {b}_{r} )$$

$${h}_{t}^{∼} = tanh ( {W}_{h} [ {g}_{r} {h}_{t - 1} , {x}_{t} ] + {b}_{h} )$$

$${g}_{z} = σ ( {W}_{z} [ {h}_{t - 1} , {x}_{t} ] + {b}_{z} )$$

$${h}_{t} = ( 1 - {g}_{z} ) {h}_{t - 1} + {g}_{z} {h}_{t}^{∼}$$

In the above formulas, Wᵣ, W_{z}, and Wₕ represent weight parameters; bᵣ, bₕ, and b_{z} represent bias parameters, which are automatically optimized by a backpropagation algorithm; hₜ represents a state of a timestamp t, hₜ₋₁ represents a state of a timestamp t-1, and hₜ˜ represents an updated timestamp state; g_{z} represents a reset gate control vector, and gᵣ represents an updated gate control vector; and σ represents the activation function, which is commonly used as the Sigmoid function, and tanh represents a tanh activation function.

The intelligent scale assessment subunit is configured to quantitatively score the rehabilitation action. This subunit uses a signal from the wearable unit as an input and outputs an intelligent quantitative score of the action. This part is completed using an LSTM network based on a self-attention mechanism. Firstly, the self-attention mechanism is used to perform attention encoding on a multielement signal (x₁-xₙ) of the wearable unit. Calculation of an attention mechanism module is as follows:

$$AS ( {x}_{i} , {x}_{j} ) = soft max ( \frac{( {W}_{q} {x}_{i} ) \cdot {( {W}_{k} {x}_{j} )}^{T}}{\sqrt{D}} )$$

$$AW ( {x}_{i} , {x}_{j} ) = \frac{exp ( AS ( {x}_{i} , {x}_{j} ) )}{{\sum }_{k = 1}^{11} exp ( AS ( {x}_{i} , {x}_{k} ) )}$$

$${x}_{j}^{′} = {\sum }_{n = 1}^{11} AW ( {x}_{i} , {x}_{j} ) \cdot {x}_{i}$$

As described above, W_{q} represents a query matrix; Wₖ represents a key matrix, which is automatically learned in a backpropagation process during network training; xᵢ and xⱼ represent input feature vectors at different time points; AS represents an attention score; AW represents an attention weight; xⱼ′ represents a reconstructed feature vector at a single time point; n and k represent loop parameters; exp represents an exponential function with e as a base; and softmax represents the activation function.

After self-attention encoding, the original signal (x₁-xₙ) is transformed into a reconstructed signal (x₁′-xₙ′), which is renamed as (z₁-zₙ). In this case, the reconstructed signal is input into the LSTM network to complete a classification task. If a single action on an original clinical scale is scored based on a k-point system (0, 1, . . . , k), the LSTM will complete a k-classification task. Calculation of the LSTM is as follows:

$${f}_{t} = σ ( {W}_{f} \cdot [ {h}_{t - 1} , {x}_{t} ] + {b}_{f} )$$

$${i}_{t} = σ ( {W}_{i} \cdot [ {h}_{t - 1} , {x}_{t} ] + {b}_{i} )$$

$${C}_{t}^{∼} = tanh ( {W}_{C} \cdot [ {h}_{t - 1} , {x}_{t} ] + {b}_{C} )$$

$${C}_{t} = {f}_{t} \times {C}_{t - 1} + {i}_{t} \times {\tilde{C}}_{t}$$

$${o}_{t} = σ ( {W}_{o} [ {h}_{t - 1} , {x}_{t} ] + {b}_{o} )$$

$${h}_{t} = {o}_{t} \times tanh ( {C}_{t} )$$

An input gate acts on a cell state Cₜ (Cₜ₋₁ represents a cell state of a previous timestamp), and determines to store which new information to the cell state. The input gate is constituted by following two parts: Input part: It is configured to construct input information and determine to add which information to the cell state as a new memory. Candidate state part: It is configured to construct a candidate cell state Cₜ˜. After the two parts are constructed, the cell state Cₜ is updated. An output gate determines the final output hₜ (hₜ₋₁ represents a hidden state of the previous timestamp). The output is completed in two steps based on a new unit state Cₜ. A first step is to determine which part of the cell state needs to be output. A second step is to control the final output. W_{f}, Wᵢ, and Wₒ represent weight parameters; b_{f}, bᵢ, b_{c}, and bₒ represent bias parameters, which are automatically optimized by the backpropagation algorithm; xₜ represents an input feature vector at a time point t; iₜ represents the input gate, and oₜ represents the output gate; and σ represents the activation function, which is commonly used as the Sigmoid function, and tanh represents the tanh activation function.

An output of the LSTM passes through a softmax layer to obtain a final probability output (p₁-pₖ), and after the one-hot encoding, a classification output can be completed, which is a final intelligent score. The intelligent scale assessment subunit not only provides an intelligent scale score, but also provides a refined scale score s. A calculation method is as follows:

$$s = {\sum }_{i = 1}^{k} i \cdot {p}_{i}$$

As described above, s represents the refined scale score, i represents the loop parameter, and pi represents a probability output.

The compensatory-movement quantitative assessment subunit includes compensation detection of a movement and quantization of a compensatory movement. In a compensatory movement detection model, probabilities of recognizing a movement parameter feature as different types (a total of seven types) of compensatory motions are determined through an internally disposed classifier, and a highest probability value and a name of a corresponding compensatory movement are used as a detection result. After the detection result shows that there is compensation, a quantization threshold interval corresponding to the movement parameter feature is determined by a compensatory-movement quantitative assessment model based on a K-nearest neighbor algorithm by a quantization threshold interval of a severity of the compensatory movement as an assessment label of a quantization category, and a quantization category corresponding to the quantization threshold interval is output as a quantitative assessment result of the compensatory movement.

The internal classifier for compensatory movement detection is a support vector machine (SVM) model. Firstly, based on a completion degree, a smoothness degree, and a fluctuation degree of a test action of a limb movement of a target patient, feature extraction is performed on limb movement data, and movement parameter features related to a trunk, a shoulder joint, and an elbow joint of the target patient are screened out to obtain the movement feature vector. In order to detect the seven types of compensatory movements present in the target patient, seven binary classification models are constructed as representatives of these types of compensatory movements. Finally, output results of seven SVM binary classifiers are integrated to detect and classify a plurality of types of compensatory movements. That is, an input of each binary classification model is a feature vector, and an output is probabilities p₀ and p₁ that a classification result is 0 or 1. If the p₁ is greater than the p₀, a highest p₁ output from the seven models corresponds to a final compensatory movement type. If the p₁ is not greater than p₀, there is no compensation.

Based on the K-nearest neighbor algorithm by the quantization threshold interval of the severity of the compensatory movement as the assessment label of the quantization category, the compensatory-movement quantitative assessment model determines the quantization threshold interval corresponding to the movement parameter feature, and outputs the quantization category corresponding to the quantization threshold interval as the quantitative assessment result of the compensatory movement.

Specifically, the quantization threshold interval of the severity of the compensatory movement is a range of each of K severity levels obtained for a same type of compensatory movement based on a severity of the compensatory movement. Quantization threshold intervals for mild compensation, moderate compensation, and severe compensation are determined by performing clustering analysis on movement parameter features of a plurality of stroke patients with this type of compensatory movement by a K-clustering algorithm. The clustering analysis is performed based on large sample data by a K-means clustering algorithm to obtain a label for dividing the severity of the compensatory movement into intervals. A K-level threshold for assessing the severity of the compensatory movement is obtained through parameter setting, where K is greater than or equal to 3. When the K is equal to 3, the quantization threshold intervals corresponding to the mild compensation, the moderate compensation, and the severe compensation are respectively a first-type threshold interval, a second-type threshold interval, and a third-type threshold interval. Afterwards, a training set and a test set are obtained through division based on a supervised machine learning algorithm, and the movement parameter feature and a multi-level assessment label are used as inputs for the compensatory-movement quantitative assessment model. A K-nearest neighbor machine learning algorithm is used to achieve quantitative assessment of the compensatory severity based on a multi-level threshold label.

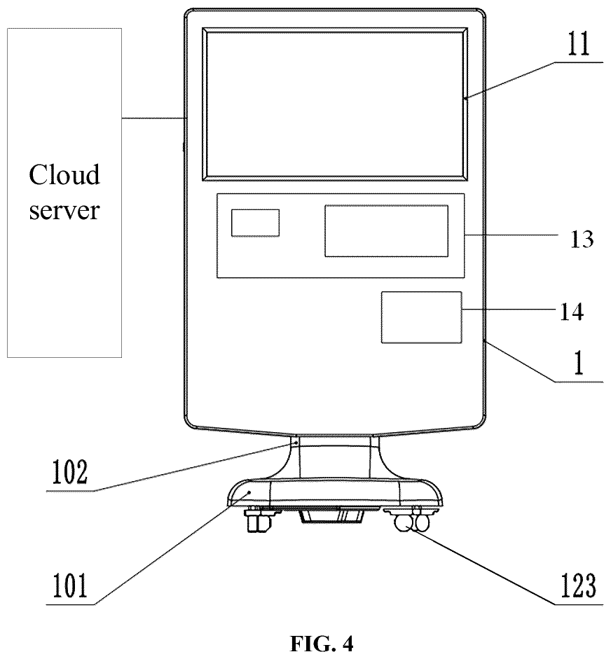

As shown in FIG. 4, the mobile unit 12 includes a roller 123, an electric push rod 122, and a driver 121 that are installed on the mobile cart 1. The driver 121 is connected to the mobile control module 113. The mobile control module 113 includes a controller for controlling the driver 121 to drive the roller 123 to move, and controlling the electric push rod 122 to stretch or retract, thereby adjusting a height of the embedded upper computer 11 to adapt to a current rehabilitation training patient.

In the embodiments, the embedded upper computer 11 is embedded into an upper part of a main body of the mobile cart 1. The mobile cart 1 further includes a base 101 and a connecting piece 102, as shown in FIG. 2. The roller 123 is disposed at a bottom of the base 101, and the electric push rod 122 is disposed inside the connecting piece 102. The embedded upper computer 11 is connected to an output end of the electric push rod 122, so as to control the height of the embedded upper computer 11 through the electric push rod 122.

It should be noted that in the embodiments, the rehabilitation training module 114 and the rehabilitation assessment module 115 constitute a rehabilitation training and assessment system. After logging into the rehabilitation training and assessment system, the rehabilitation training patient can select rehabilitation training and assessment content as required. If the rehabilitation training content is selected, the rehabilitation training patient needs to wear the rehabilitation training glove 201 and the somatosensory sensor 202 first, and then selects a rehabilitation training function in a displayed window. In the rehabilitation training function, many rehabilitation training games are loaded, and the rehabilitation training patient can select a difficulty of a corresponding rehabilitation training game as required. For a patient who uses the device for the first time, the system also loads a teaching video. The rehabilitation training patient can learn a usage method based on the video. This can help a user better use the device and further improve user experience. The rehabilitation training game is an interesting small game that is closely related to daily life and is used to fully train the upper limb, the lower limb, and the finger of the patient, accelerating rehabilitation progress of the patient.

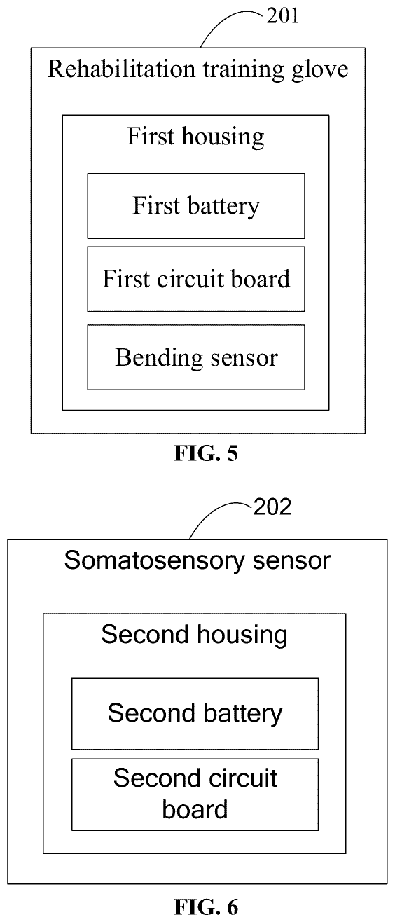

As shown in FIG. 5, the rehabilitation training glove 201 includes a glove body and a first housing installed on the glove body. The first housing is equipped therein with a first battery, a first circuit board, and a bending sensor, and a first indicator light is installed on the first housing. In the embodiments, the glove body is made of a nylon material, and a mouth of the glove is elastic to enhance a comfort level. There are various types of gloves suitable for different populations. The first battery is a lithium battery whose model is Zhongshun 602035, which provides power support for other components on the glove. The bending sensor selected is an FS-L-0055-253-ST bending sensor, which is configured to measure relevant data of the rehabilitation training patient during training. The first circuit board is configured to send the data of the bending sensor to the data collection module 111 through an integrated wireless transmission module. In addition, the first circuit board is further configured to control flashing of the first indicator light. When a corresponding channel device of the rehabilitation training glove 201 is running normally, the first circuit board controls the indicator light to be green (or flash). When the corresponding channel device has no data, the indicator light is gray. When the corresponding channel device receives data abnormally, the indicator light is red.

As shown in FIG. 6, the somatosensory sensor 202 includes a bandage and a second housing installed on the bandage. The second housing is equipped therein with a second circuit board and a second battery, and a second indicator light is installed on the second housing. In the embodiments, there may be a plurality of somatosensory sensors 202, which are mainly configured to collect movement data of the upper and lower limbs. The bandage may be of different sizes as required to fix the second housing and its internal second circuit board and second battery in corresponding positions. The second battery may be of a same model as the first battery to provide power support. As required, the second housing is equipped with a corresponding sensor connected to the second circuit board, such as an attitude sensor. The second circuit board is configured to control flashing of the second indicator light and send data of the sensor to the data collection module 111 through the integrated wireless transmission module.

It can be understood that in the embodiments, in addition to the rehabilitation training glove 201 and the somatosensory sensor 202, other wearable devices or rehabilitation training instruments such as a walking machine and a rehabilitation training helmet can also be used.

Preferably, the data collection module 111 is connected to a receiver 1111 and a camera 1112. The receiver 1111 is wirelessly connected to both the rehabilitation training glove 201 and the somatosensory sensor 202 to receive movement data from the rehabilitation training glove 201, the somatosensory sensor 202, and other devices. The camera 1112 is configured to collect the movement data by capturing an action of the rehabilitation training patient. In the embodiments, the camera 1112 may be a built-in camera 1112 of the embedded upper computer 11, or an externally connected camera 1112. There is no special requirement for a model of the camera 1112, provided that the camera 1112 is compatible with the embedded upper computer 11 and the rehabilitation training and assessment system.

Preferably, in the embodiments, the embedded upper computer 11 can be connected to a cloud server through a network. The cloud server stores an exercise prescription. The cloud server is configured to download the exercise prescription or upload the rehabilitation training data. In a practical application, the rehabilitation training patient can also be connected to a terminal device in a hospital through the cloud server, enabling remote online guidance and diagnosis by a doctor.

As shown in FIG. 4, the device further includes a human-machine interaction column 13. The human-machine interaction column 13 is connected to the embedded upper computer 11 in a wired or wireless manner. The human-machine interaction column 13 is equipped with a card reading area and a button area. The card reading area is configured to recognize an identity of the rehabilitation training patient, and the button area is equipped with a plurality of buttons to assist in the scene training game. Before using the device for the rehabilitation training, the rehabilitation training patient needs to swipe his/her ID card or identification card in the card reading area. The embedded upper computer 11 implements identity authentication through the ID card or the identification card, and then the rehabilitation training patient can enter the rehabilitation training and assessment system and play the rehabilitation training game through the button in the button area. It can be understood that a management system for the rehabilitation training patient can be established based on the human-machine interaction column 13, and a record can be created for each rehabilitation training and assessment of the patient, keeping track of the rehabilitation progress at any time. This not only improves the user experience, but also reduces a burden on staff through detailed record management, making an entire rehabilitation training process more standardized.

Preferably, the device further includes a power module 14. The power module 14 includes a magnetic ring, a transformer, a filter, a data line, and the like, and is configured to provide a power supply and process a power signal. A V18004, V18005 or V18007 magnetic ring is selected to achieve electromagnetic shielding. A RSEN-2006L EMC filter is selected to process the power signal. A H0128-823-0250 isolation transformer is selected. The magnetic ring, the transformer, and the filter are all disposed in a cavity inside the base 101, which makes the base 101 heavy and easy to maintain stability of the embedded upper computer 11 connected to the base 101 during movement. The power supply and the embedded upper computer 11 need to be connected between the components as needed, and details are not described herein again.

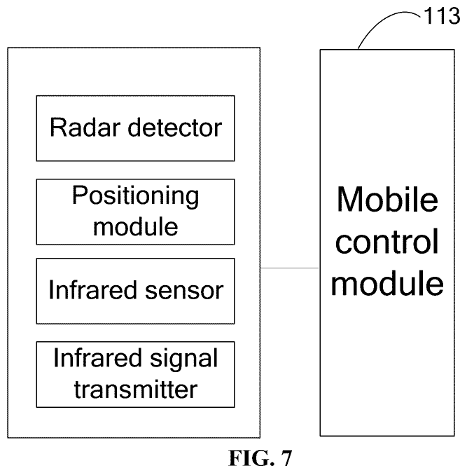

As shown in FIG. 7, the mobile control module 113 is connected to a radar detector. The radar detector is configured to detect the position of the rehabilitation training patient. The mobile control module 113 is configured to control the driver 121 and the electric push rod 122 to work. In the embodiments, the radar detector may be a SENKYLASER SK-C10 2D laser scanning radar, and the mobile control module 113 achieves autonomous positioning and mapping by a Gmapping algorithm. It can be understood that the device further includes a global positioning system (GPS) positioning module for determining a current position of the mobile cart 1. Then, a surrounding environment is scanned through the radar detector, and the position of the rehabilitation training patient is determined. Finally, through the driver 121, the mobile control module 113 controls the roller 123 to move towards a direction of the rehabilitation training patient. In the embodiments, there are four rollers 123, and the four rollers 123 are disposed circumferentially at a bottom of the mobile cart 1. The driver 121 uses two motors. One of the motors is connected to two rollers 123, and the controller controls forward and reverse rotation of the motor to drive the roller 123 to rotate, thereby enabling the mobile cart 1 to move forward and backward. The other motor is connected to the other two rollers 123, and the controller controls forward and reverse rotation of the other motor to drive the other two rollers 123 to rotate, thereby enabling the mobile cart 1 to move left and right. The method of controlling the device to move forward, backward, left, and right through the motor is the prior art and similar to a method of controlling a toy car to move forward, backward, left, and right through a handle. The controller may be a remote control. The remote control is connected to the motor through Wi-Fi. The remote control is equipped with front, back, left, and right buttons, and can be used to control the device to move forward, backward, left, and right.

It should be noted that the driver 121 is driven by a servo motor, and its model may be HC-KFS, HC-MFS, HC-SFS, HC-RFS, or HC-UFS series. An output terminal of the driver 121 is connected to the roller 123, and the driver 121 is controlled by the mobile control module 113 to enable the cart 1 to move. The four rollers 123 and the two drivers 121 are disposed to better enable the cart 1 to move forward, backward, left, and right. The four rollers 123 are disposed at the bottom of the base 101. One of the drivers 121 is connected to two rollers 123, and the forward and reverse rotation of the driver 121 is controlled to enable the cart 1 to move forward and backward. The other driver 121 is connected to the other two rollers 123, and the forward and reverse rotation of the driver 121 is controlled to enable the cart 1 to turn left and right.

Due to varying heights of different users, in order to achieve automatic height adjustment of the device, an infrared sensor connected to the mobile control module 113 is disposed on a top of the mobile cart 1. During use, the patient only needs to wear a corresponding infrared signal transmitter. During the use, the mobile control module 113 controls the electric push rod 122 to work within a certain range, such that the embedded upper computer 11 moves within a certain height range. When the infrared sensor and the infrared signal transmitter are at a same height, the infrared sensor receives an infrared signal emitted by the infrared signal transmitter. At this time, the mobile control module 113 controls the electric push rod 122 to stop working, and a most suitable height for the current patient is obtained.

In other embodiments, the device can achieve manual height adjustment. A lifting and lowering program of the electric push rod 122 is integrated into the mobile control module 113. The embedded upper computer 11 can be lifted or lowered by opening a lifting and lowering control interface of the electric push rod 122 in the mobile control module 113 and clicking an up option or a down option.

In other embodiments, the mobile control module 113 includes a remote control. The remote control is connected to the embedded upper computer 11 through Wi-Fi. The mobile control module 113 controls the electric push rod 122 through a corresponding up or down button on the remote control, thereby lifting or lowering the embedded upper computer 11.

In the embodiments, the data collection module 111 collects an original limb movement signal of the rehabilitation training patient through the receiver 1111 and the camera 1112, and sends the limb movement signal to the data processing module 112 for processing. The data processing module 112 first preprocesses the limb movement signal to eliminate interference and influence of system noise. Next, it is necessary to perform the feature extraction on the original limb movement signal. In the embodiments, the feature extraction is performed from perspectives of a completion degree, smoothness, and the like of completing the action by the patient to obtain a one-dimensional feature vector of the rehabilitation training action. Finally, it is necessary to use a multi-source fusion algorithm to quantitatively assess features of a plurality of dimensions, and drive (control) and provide a human-machine interaction feedback for a task and a role in a virtual reality scene based on a result.

The rehabilitation assessment module 115 has established an action recognition model for the wearable device based on a sequential network. After completing the rehabilitation training action, the patient needs to assess quality of the training action. After the one-dimensional feature vector that is of the rehabilitation training action and processed by the data collection module 111 is obtained, a training action assessment model is established using an LSTM scale assessment network based on the attention mechanism and a compensation detection network based on machine learning. In action assessment, a prediction process is an assessment process (providing an action score and a compensation score), and the data is included in the training set to update the training model, such that the model has a self-learning function. On a basis of the model, visualized quantitative assessment parameters are designed by nonlinear dynamic tools such as a Poincare difference scatter plot, a maximum Lyapunov exponent, and a distribution entropy, such that the quantitative assessment model can provide a visual quantitative score for the training action.

The foregoing descriptions are merely optimal specific implementations of the present disclosure, but are not intended to limit the protection scope of the present disclosure. Any variation or replacement within the technical scope disclosed in the present disclosure shall fall within the protection scope of the present disclosure. Therefore, the protection scope of the present disclosure should be subject to the protection scope of the claims.

1. An intelligent rehabilitation device, comprising a mobile cart, wherein
the mobile cart is equipped with an embedded upper computer, a mobile unit, and a wearable unit, and both the mobile unit and the wearable unit are communicatively connected to the embedded upper computer;
the embedded upper computer comprises a rehabilitation training module, a rehabilitation assessment module, a data collection module, a data processing module, a mobile control module, and a storage module;
the rehabilitation training module is configured to provide a scene training game designed based on a rehabilitation action, and the scene training game is used to train an upper limb, a lower limb, and a hand of a rehabilitation training patient;
the rehabilitation assessment module is configured to assess movement functions of the upper limb, the lower limb, and the hand of the rehabilitation training patient;
the data collection module is configured to collect information of the rehabilitation training patient, comprising feature vectors corresponding to a plurality of dimensions of the patient, wherein each feature vector is used to indicate patient information of each dimension, and the dimensions comprise a medical record, a lifestyle habit, an environmental factor, and a rehabilitation exercise of the patient;
the data processing module is configured to: predict an impact of the patient information of each dimension on rehabilitation of the patient based on the feature vector of each dimension, obtain a first prediction vector corresponding to the feature vector of each dimension, and perform feature concatenation on each first prediction vector to obtain a target vector, wherein the first prediction vector is used to indicate the impact of the patient information of each dimension on the rehabilitation of the patient; predict, based on the target vector, a rehabilitation effect corresponding to the patient information of each dimension, and obtain a second prediction vector, wherein the second prediction vector is used to indicate the rehabilitation effect corresponding to the patient information of each dimension; and generate prescription information for the rehabilitation of the patient based on the second prediction vector, wherein the prescription information comprises medication information and a medication dosage;
the mobile control module is configured to control, by obtaining a position of the rehabilitation training patient and measuring a distance from the rehabilitation training patient, the mobile cart to move and adjust a height;
the mobile unit comprises a roller, an electric push rod, and a driver that are disposed on the mobile cart, wherein the driver is connected to the mobile control module, and through the driver, the mobile control module drives the roller to move and adjust a position, and controls the electric push rod to adjust a height; and
the wearable unit comprises a rehabilitation training glove and a somatosensory sensor, wherein both the rehabilitation training glove and the somatosensory sensor are configured to perform rehabilitation training through the rehabilitation training module and send rehabilitation training data to the data collection module.

2. The intelligent rehabilitation device according to claim 1, wherein the data collection module comprises a receiver and a camera, wherein the receiver is wirelessly connected to both the rehabilitation training glove and the somatosensory sensor;
the receiver is configured to receive movement data from the rehabilitation training glove and the somatosensory sensor, and the camera collects the movement data by capturing an action of the rehabilitation training patient; and
the data processing module is further configured to process the movement data based on multi-source information fusion.

3. The intelligent rehabilitation device according to claim 1, wherein the intelligent rehabilitation device further comprises:
a human-machine interaction column equipped with a card reading area and a button area, wherein the card reading area is configured to recognize an identity of the rehabilitation training patient, and the button area is equipped with a plurality of buttons to assist in the scene training game; and
a power module, wherein the power module comprises a magnetic ring, a transformer, a filter, and a data line, and is configured to provide a power supply and process a power signal;
the embedded upper computer is also communicatively connected to a cloud server;
the cloud server stores an exercise prescription; and
the embedded upper computer is further configured to download the exercise prescription or upload the rehabilitation training data through the cloud server.

4. The intelligent rehabilitation device according to claim 1, wherein the rehabilitation training glove comprises a glove body and a first housing installed on the glove body; and
the first housing is equipped therein with a first battery, a first circuit board, and a bending sensor, and a first indicator light is installed on the first housing.

5. The intelligent rehabilitation device according to claim 1, wherein the somatosensory sensor comprises a bandage and a second housing installed on the bandage; and
the second housing is equipped therein with a second circuit board and a second battery, and a second indicator light is installed on the second housing.

6. The intelligent rehabilitation device according to claim 1, wherein the rehabilitation assessment module comprises an assessment assistance unit and an assessment unit, wherein the assessment unit comprises an intelligent scale assessment subunit and a compensatory-movement quantitative assessment subunit;
the assessment assistance unit is configured to complete a task of recognizing a rehabilitation assessment action and obtain a recognition result; and
the assessment unit is configured to obtain a corresponding assessment result based on the recognition result.

7. The intelligent rehabilitation device according to claim 1, wherein the embedded upper computer is interconnected with a terminal through a network, and configured for remote medical diagnosis or online rehabilitation training guidance.

8. The intelligent rehabilitation device according to claim 1, wherein the mobile control module is connected to a radar detector and a positioning module;
the radar detector is configured to detect the position of the rehabilitation training patient; and
the positioning module is configured to determine a current position of the mobile cart.

9. The intelligent rehabilitation device according to claim 1, wherein the mobile cart comprises a base and a connecting piece; and
the roller is disposed at a bottom of the base, the electric push rod is disposed inside the connecting piece, and the embedded upper computer is connected to a top of the electric push rod.

10. The intelligent rehabilitation device according to claim 1, wherein the mobile control module is connected to an infrared sensor and an infrared signal transmitter, and when the intelligent rehabilitation device is in use, position and height adjustment is achieved through the infrared sensor, the infrared signal transmitter, and the mobile control module.
